(19) United States
(12) Patent Application Publication
(10) Pub. No.: US 2013/0245492 A1
(43) Pub. Date: Sep. 19, 2013

(54) Dispensing Device for Prefilled Syringe

(71) Applicants: Karl-Michael Klenk, Mesa, AZ (US); Curtis P. Hamann, Paradise Valley, AZ (US); Bryan Richard Falk, Chandler, AZ (US)

(72) Inventors: Karl-Michael Klenk, Mesa, AZ (US); Curtis P. Hamann, Paradise Valley, AZ (US); Bryan Richard Falk, Chandler, AZ (US)

(73) Assignee: SMARTHEALTH, INC., Phoenix, AZ (US)

(21) Appl. No.: 13/421,109

(22) Filed: Mar. 15, 2012

(51) Int. Cl. A61B 5/00; B23P 17/04

(52) U.S. Cl. 600/556; 29/428

(57) ABSTRACT

A driver for engaging and dispensing fluid from a sealed, prefilled syringe is provided. The driver includes a body and a rotating actuator configured to engage a plunger of the prefilled syringe. The driver is configured such that rotation of the rotating actuator relative to the body imparts both rotational movement and axial movement to a plunger of the prefilled syringe resulting in ejection of fluid from the prefilled syringe.

### BACKGROUND OF THE INVENTION

The present invention relates generally to the field of prefilled syringes. The present invention relates specifically to a dispensing device or driver for dispensing fluid from a prefilled syringe.

Prefilled syringes are used in a variety of areas, including for medical tests, therapeutic uses, and scientific uses. Prefilled syringes typically have a syringe body or syringe barrel and a plunger. The plunger head seals to the inner surface of the barrel forming a sealed cavity or chamber that holds a fluid, such as a medical test or therapeutic substances. The plunger includes a shaft coupled at one end to the plunger head, and at the other end, the shaft is coupled to a plunger top or disc, sized to facilitate engagement by a user's fingers or thumb. Pushing the plunger disc forces the plunger head toward a dispensing opening located through the syringe body resulting in the dispensing or ejection of fluid from the syringe body.

Such prefilled syringes are used in many areas. For example, prefilled syringes may be used to hold allergen test substances. Typically, each prefilled syringe holds a volume of a single allergen test substance that may be used for multiple allergy tests for multiple patients. For example, such allergen test prefilled syringes may originally hold 5 milliliters of test substance. During an allergy test, a health care worker typically will dispense a small volume (e.g., less than 100 microliters) of test substance from the prefilled syringe into a receiving chamber that has been attached to the skin of a patient receiving an allergy test. The receiving chamber holds the allergen test substance in contact with the patient's skin, and the test area of skin is monitored for allergic reaction. Because only a small amount of the test substance is used for a single test, the prefilled syringe is used for multiple allergy tests for multiple patients. A typical health care facility will have a number of different prefilled syringes, each one holding a different allergen test substance.

### BRIEF SUMMARY OF THE INVENTION

One embodiment of the invention relates to a dispensing device or driver configured to engage a prefilled syringe to improve the precision of the volume of fluid dispensed from the prefilled syringe. The driver is removably engageable with the prefilled syringe such that the dispensing device may be used with multiple prefilled syringes. Further, the various structures of the driver are also configured to only engage with exterior or dry portions of the prefilled syringe (i.e., portions not in contact with the substance of the prefilled syringe) allowing the driver to be used with prefilled syringes holding different allergen test substances. The driver is configured to impart both axial and rotational movement to the plunger of the prefilled syringe allowing for more precise dispensing of the substance from the prefilled syringe as compared to direct operation of the plunger that the prefilled syringe was originally equipped with.

Another embodiment of the invention relates to a driver for engaging a sealed, prefilled syringe. The prefilled syringe includes a syringe body, a flange extending from an upper end of the syringe body, and a plunger received within the syringe body. The driver includes a handle body and a shaft. The handle body includes a first end, a second end opposite the first end, a central passage extending through the body from the first end to the second end, and an attachment structure located at the second end. The attachment structure is configured to rigidly attach to the flange of the prefilled syringe such that the syringe body is held in a fixed position relative to the handle body. The driver includes a threaded inner surface located within the central passage of the handle body. The shaft of the driver is configured to extend through the central passage of the handle body. The shaft includes a first end, a second end opposite the first end, an engagement section adjacent the second end and a threaded outer surface portion configured to engage the threaded inner surface. The engagement section includes a first slot formed in the shaft configured to receive a portion of the syringe plunger. The driver is configured such that rotation of the shaft relative to the handle body causes advancement of the shaft through the central passage imparting both rotational movement and axial movement to the plunger of the prefilled syringe.

Another embodiment of the invention relates to a system for dispensing fluid from a prefilled syringe. The system includes a prefilled syringe and a driver. The prefilled syringe includes a syringe body having an upper end, a lower end opposite the upper end, and a central cavity extending from the upper end to the lower end. The prefilled syringe includes a plunger including a plunger head received within the central cavity of the syringe body and a plunger shaft coupled to the plunger head. The plunger shaft extends from the plunger head toward the upper end. The plunger head forms a seal within the central cavity defining a contents chamber. The prefilled syringe includes a fluid located within the contents chamber. The driver includes a driver body rigidly attached to the upper end of the prefilled syringe such that the syringe body is held in a fixed position relative to the driver body. The driver includes a rotating actuator engaging the plunger of the prefilled syringe configured to impart both rotational and axial movement to the plunger to dispense the fluid from the prefilled syringe.

Another embodiment of the invention relates to a method of modifying a prefilled syringe. The method includes the step of providing a prefilled syringe. The prefilled syringe includes a syringe body including a first end, a second end opposite the first end, and a central cavity extending from the first end to the second end. The prefilled syringe includes a plunger including a plunger seal, plunger top and a plunger shaft extending between the plunger seal and the plunger top. The prefilled syringe includes a fluid located within the central cavity. The method includes the step of providing a driver including a rotating actuator, and the driver is configured to impart both rotational and axial movement to the plunger to dispense the fluid from the prefilled syringe. The method includes the step of cutting the plunger shaft at a position between the plunger seal and the plunger top and removing the portion of the plunger shaft above the cut including the plunger top. The method includes coupling the rotating actuator of the driver to the plunger.

Other aspects, objectives and advantages of embodiments of the invention will become more apparent from the following detailed description when taken in conjunction with the accompanying drawings.

### BRIEF DESCRIPTION OF THE DRAWINGS

This application will become more fully understood from the following detailed description, taken in conjunction with the accompanying figures, wherein like reference numerals refer to like elements in which:

FIG. 1 is a perspective view of a dispenser according to an exemplary embodiment;

FIG. 2A is an exploded view of the dispenser of FIG. 1 according to an exemplary embodiment;

FIG. 2B is a perspective view of a prefilled syringe according to an exemplary embodiment;

FIG. 2C is a perspective view of a prefilled syringe according to an exemplary embodiment;

FIG. 2D is a detailed perspective view of a handle body of the dispenser of FIG. 1 according to an exemplary embodiment;

FIG. 2E is a detailed perspective view of a shaft and indicator of the dispenser of FIG. 1 according to an exemplary embodiment;

FIG. 3 is a detailed view showing the connection between a driver and prefilled syringe according to an exemplary embodiment;

FIG. 4 is a sectional view showing a handle body of a driver according to an exemplary embodiment;

FIG. 5A is a perspective view of a sleeve assembly according to an exemplary embodiment;

FIG. 5B is an exploded view of the sleeve assembly of FIG. 5A according to an exemplary embodiment;

FIG. 5C is a perspective view of a sleeve assembly according to another exemplary embodiment;

FIG. 6A is a perspective view of a dispenser in a first position according to an exemplary embodiment;

FIG. 6B is a perspective view of the dispenser of FIG. 6A in a second position according to an exemplary embodiment; and

FIG. 7 is flow-diagram showing modification of a prefilled syringe is shown according to an exemplary embodiment.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Referring generally to the figures, a dispensing device or driver configured to engage a prefilled syringe is shown according to exemplary embodiments. Generally, the driver discussed herein is a device separate from the prefilled syringe that is configured to engage the preexisting plunger of a prefilled syringe and functions to impart both axial and rotational motion to the plunger of the prefilled syringe in a manner that provides for a precise amount of fluid to be dispensed from the prefilled syringe. In particular, the rotational movement of the plunger acts to overcome static friction between the plunger and the inner surface of the syringe in a manner that limits or prevents sudden axial motion, and thereby, acts to provide for increased dispensing accuracy from a prefilled syringe as compared to manual operation of the standard push-actuated plunger of the prefilled syringe.

The movement of the prefilled syringe plunger provided by the driver is precisely controllable by the user of the driver such that the user can accurately dispense very small volumes (e.g., less than 30 microliters) of substance from the prefilled syringe as needed from a particular application. The driver is configured to be non-permanently attached to the prefilled syringe so that the driver may be used with multiple prefilled syringes. In addition, the driver is configured to engage only dry (i.e., non-content contacting) surfaces of the prefilled syringe, and thus, preventing contamination between different prefilled syringes as the driver is used between different prefilled syringes with different contents. The driver is also configured to provide one or more indication (e.g., audible, tactile, and/or visual, etc.) that the desired amount of fluid has been dispensed from the prefilled syringe. In the specific embodiments shown in the figures, the driver is configured to be easily attached to the prefilled syringe and is configured to be easily operated manually by the user to dispense fluid.

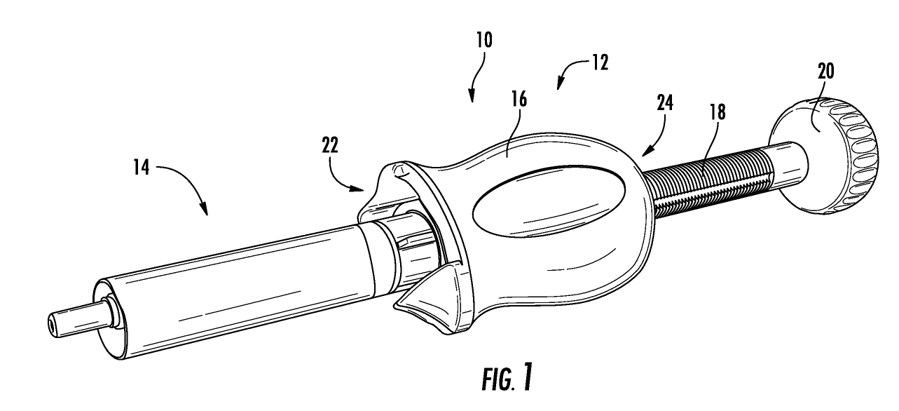

Referring to FIG. 1, a dispensing system, shown as dispenser 10, configured to precisely dispense a volume of liquid material is shown according to an exemplary embodiment. Dispenser 10 includes a driver, shown as plunger driver 12, and a prefilled syringe 14. Plunger driver 12 includes a body, shown as handle body 16, a rotating actuator, shown as driver shaft 18, and a knob, shown as end knob 20. Generally, handle body 16 includes a lower or front end 22 coupled to prefilled syringe 14, and as will be discussed in more detail below, driver shaft 18 passes through an upper or rear end 24 of handle body 16 to engage the plunger of prefilled syringe 14.

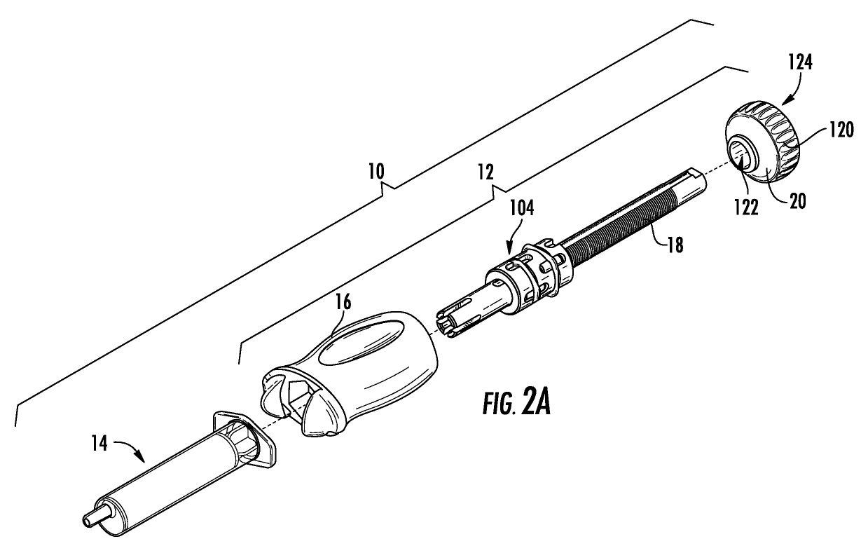

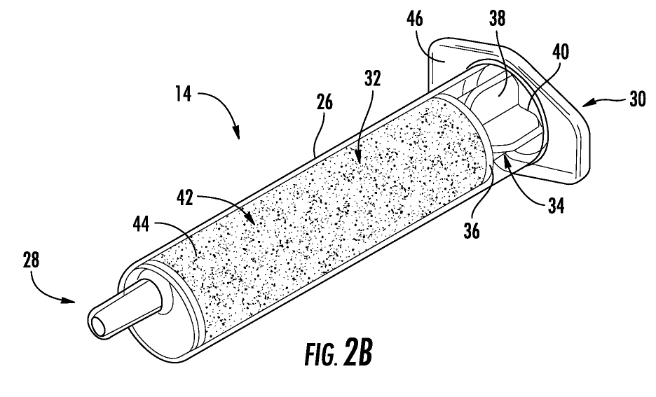

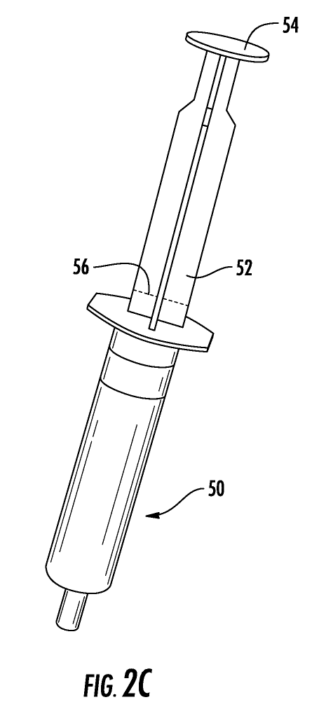

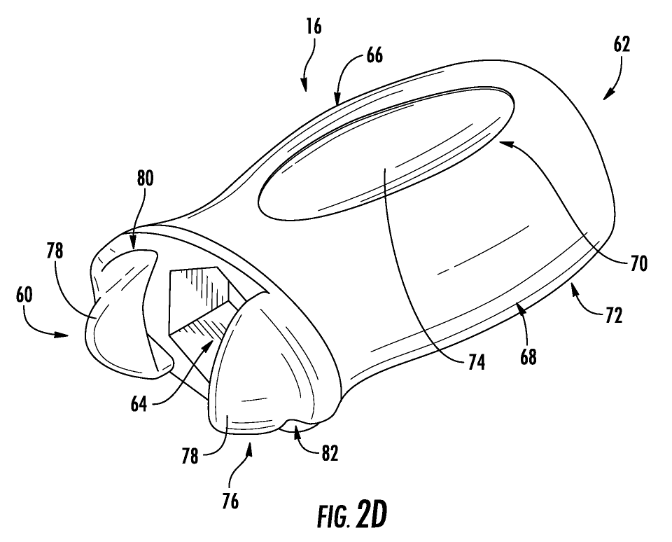

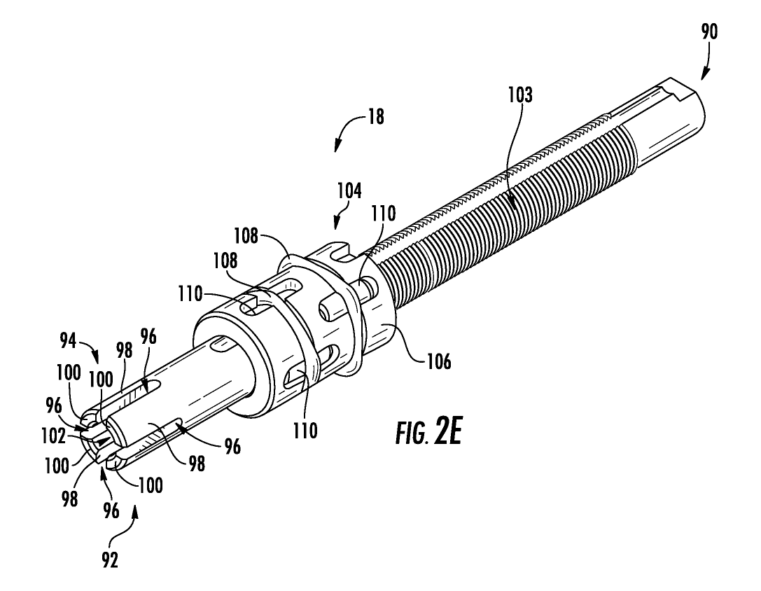

Referring to FIG. 2A, an exploded view of dispenser 10 is shown according to an exemplary embodiment. As generally shown in FIG. 2A, to assemble dispenser 10, shaft 18 extends through handle body 16 to engage the plunger of prefilled syringe 14. Handle 16 rigidly engages the upper end of the outer body of prefilled syringe 14, and knob 20 mounts to the upper end of shaft 18. Rotation of shaft 18 may be applied by the user via knob 20 which in turn causes rotation and axial advancement of the plunger of prefilled syringe 14 causing the ejection of fluid from syringe 14. Each of the components of dispenser 10 will be discussed in more detail below.

Referring to FIG. 2B a detailed view of prefilled syringe 14 is shown. Prefilled syringe 14 is shown having a syringe body 26 having a lower, dispensing end 28 and an upper end 30. Syringe body 26 includes a central cavity or channel 32 formed within the syringe body that extends from upper end 30 to dispensing end 28. Syringe 14 includes a plunger 34 having a plunger head 36 and a plunger shaft 38. Plunger shaft 38 includes a lower end coupled to plunger head 36 and an upper end 40. Plunger head 36 forms a seal with the inner surface of syringe body 26 defining a contents chamber 42 of prefilled syringe 14 located within central cavity 32 between dispensing end 28 and plunger head 36. A fluid or substance 44 is contained within chamber 42. In one embodiment, substance 44 is an allergen test substance that may be applied to a patient's skin as part of an allergy test. In one such embodiment, prefilled syringe 14 is a 5 milliliter prefilled syringe. In other embodiments, other medical or therapeutic substances may be contained within chamber 42.

Prefilled syringe 14 includes a flange, shown as syringe head 46, located at upper end 30 and that extends radially outward from and is substantially perpendicular to syringe body 26. In the embodiment shown, syringe head 46 is shaped as an elongated polygon having a major axis and a minor axis such that the syringe head 46 extends further from syringe body 26 along the major axis than along the minor axis. As explained in more detail below, syringe head 46 provides mounting surfaces that allows driver 12 to be coupled to prefilled syringe 14.

In the embodiment shown in FIG. 2B, the length of plunger shaft 38 is relatively short compared to the length of cavity 32 or to the length of syringe body 26. In various embodiments the length of plunger shaft 38 is less than half the length of cavity 32, preferably is less than a third of the length of cavity 32 and more preferably is less than a quarter of the length of cavity 32. In the embodiment shown, plunger shaft 38 is formed of two substantially planar pieces of material positioned perpendicular to each other such that plunger shaft 38 has a substantially cross-shaped cross-section. Further, upper end 40 of plunger shaft 38 is not coupled to a radially extending planar plunger top (like the one shown in FIG. 2C), and the cross-sectional shape and area of upper end 40 of plunger shaft 38 is substantially the same as the cross-shaped cross-section of lower portions of plunger shaft 38. Further, FIG. 2B shows prefilled syringe 14 holding the maximum amount of substance 44 (i.e., that is before any substance 44 has been dispensed), and in this embodiment, plunger shaft 38 is sized such that upper end 40 of plunger shaft 38 is substantially coplanar with the upper most surface of syringe head 46 when the prefilled syringe is full. As the plunger is advanced during dispensing of fluid from prefilled syringe 14, upper end 40 of plunger shaft 38 moves axially downward within cavity 32.

In one embodiment, prefilled syringe 14 is a standard prefilled syringe that is modified for engagement with driver 12. Referring to FIG. 2C, a typical, prefilled syringe 50 is shown. Prefilled syringe 50 is the type which a user's fingers or thumbs are used to directly actuate the syringe plunger 52 via application of force to a plunger top or thumb disc 54. In one embodiment, the shaft of syringe plunger 52 is cut or severed after syringe 50 has been filled, resulting in prefilled syringe 14 having the shorter plunger shaft 38 shown in FIG. 2B. In such embodiments, prefilled syringe 50 is purchased by a user (e.g., doctor's office, hospital, clinician, etc.). The user then cuts the shaft of plunger 52 at a position, shown by line 56, along its length between the plunger's upper and lower ends to produce plunger shaft 38. In one such embodiment, the shaft of plunger 52 is cut so that the cut or severed end of shaft 38 is substantially coplanar with the upper most surface of syringe head 46. In one embodiment, driver 12 and prefilled syringe 14 may be included in a kit along with a cutter configured to cut the material of plunger 52. In another embodiment, plunger 52 of syringe 50 may include a weakness, a score, or other frangible section that facilitates removal of the upper portion of plunger 52. In another embodiment, plunger 52 may include an indicia, for example a guide line, that traverses the width of the shaft of plunger 52 that provides an indication of where plunger 52 is to be cut. In other embodiments, driver 12 is used with a syringe, such as syringe 14, that is constructed with a shortened plunger shaft 38 as shown in FIG. 2B (e.g., prefilled syringe 14 may be purchased with a shortened plunger shaft 38).

Referring back to FIG. 2A, dispenser 10 includes driver 12, and driver 12 includes handle body 16, threaded shaft 18 and knob 20. Referring to FIG. 2D a detailed view of handle body 16 is shown. Handle body 16 includes a lower end 60, an upper end 62 and a central channel or passage 64 extending through handle body 16 from the first end to the second end. As will be discussed in more detail below, threaded shaft 18 extends through central passage 64 of handle body 16 to engage with plunger 34 of prefilled syringe 14.

In the embodiment shown, handle body 16 is shaped and sized to provide for an easy and comfortable grip within a hand of a user. Specifically, handle body 16 is elongated in the direction of passage 64 such that the length of handle body 16 is greater than both the width and thickness of the handle. Further, the opposing lateral surfaces 66 and 68 are curved surfaces extending first radially outward as the distance from upper end 62 increases and then curve radially inward from a maximum width as the distance from upper end 62 increases further. Handle body 16 includes a front face 70 extending between lateral surfaces 66 and 68 on one side of handle body 16 and a rear face 72 (opposite front face 70 in the orientation of FIG. 2D) extending between lateral surfaces 66 and 68 on the other side of handle body 16. It should be understood that FIG. 2D shows front face 70 and that rear face 72 is substantially a mirror image of front face 70. Front face 70 and rear face 72 includes one or more label area 74.

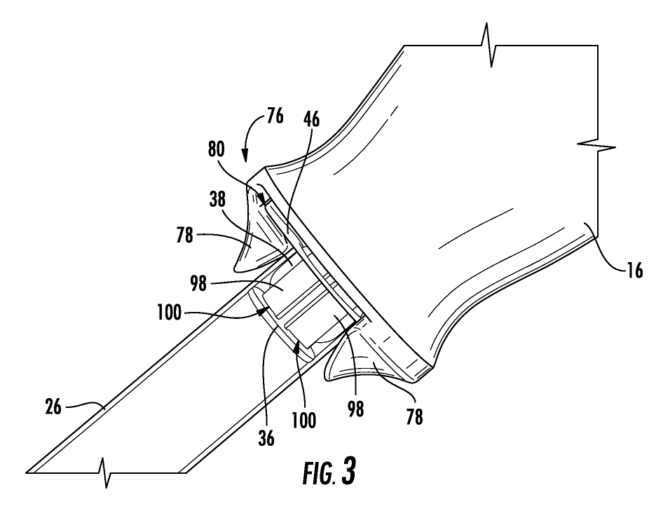

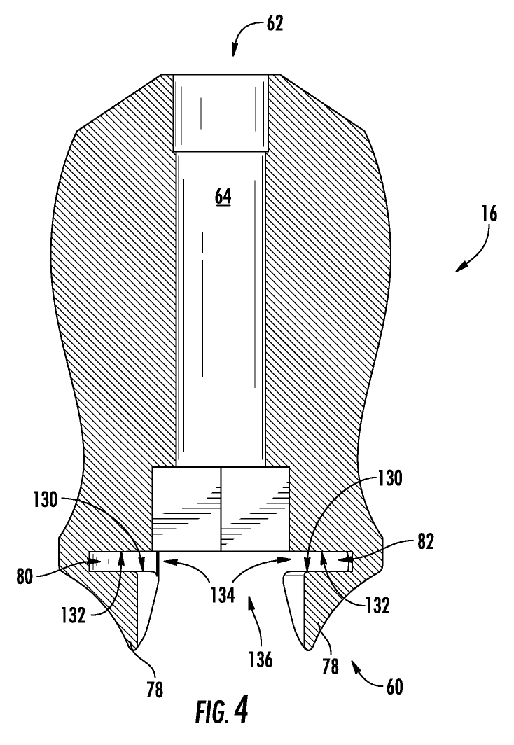

Handle body 16 includes an attachment structure 76 located at lower end 60. As explained in more detail below, attachment structure 76 is configured to rigidly engage syringe body 26 (i.e., engagement such that handle body 16 and syringe body 26 remain substantially fixed relative to each other during movement of syringe plunger 34 by driver 12). Generally, attachment structure 76 includes a flared collar 78 surrounding the lower opening to passage 64. As shown in FIG. 1 and FIG. 3, when coupled to prefilled syringe 14, flared collar 78 partially surrounds an upper portion of syringe body 26. As explained in more detail below, attachment structure 76 includes a first slot 80 formed through front surface 70 and a second slot 82 (also shown in FIG. 4) formed through rear surface 72. Slots 80 and 82 allow entry of the syringe head 46 into attachment structure 76 during attachment of driver 12 to syringe 14.

Referring to FIG. 2E, a detailed view of threaded shaft 18 is shown. Threaded shaft 18 includes a upper end 90 and a lower end 92. Upper end 90 is configured to be coupled to knob 20. In one embodiment, upper end 90 includes a post that is press-fit inside knob 20. In another embodiment, upper end 90 includes a thread bore for receiving a screw coupling knob 20 to shaft 18.

Shaft 18 includes an engagement structure or section, shown as pronged end 94 located adjacent lower end 92. The engagement structure includes a least one slot configured to receive at least a portion of plunger shaft 38 of prefilled syringe 14. In the embodiment of FIG. 2E, the engagement structure includes four slots 96 evenly positioned at 90 degree intervals from each other and defining a void 102 that has a cross-sectional shape substantially matching the cross-sectional shape of plunger shaft 34. Slots 96 and void 102 define four fingers or prongs 98 located adjacent lower end 92. Each prong 98 includes an axial facing end surface 100. As discussed below regarding FIG. 3, prongs 98 are shaped to surround plunger shaft 38 and axial facing end surfaces 100 are configured to engage an upward facing surface of plunger head 36. The engagement between pronged end 94 and plunger head 36 allows movement of threaded shaft 18 to cause movement of plunger 34 resulting in dispensing of an amount of fluid proportional to the axial movement of plunger 34.

A portion of the outer surface of shaft 18 includes threads 103. Coupled to shaft 18 is an indicator, shown as sleeve assembly 104. Sleeve assembly includes a cylindrical sleeve 106, a pair of biasing elements, shown as bands 108, and a plurality of rigid bodies, shown as pins 110. Each band 108 circumscribes a portion of cylindrical sleeve 106 engaging four of the eight pins 110. Bands 108 are made from an elastic material that is configured to urge or bias pins radially inward toward shaft 18.

Threaded shaft 18 extends through and is coupled to cooperative threading located on the inner surface of a bore extending through the center of sleeve 106. Shaft 18 includes a depression, shown as axial groove 112, that cooperates with sleeve assembly 104 to provide an indication of the amount of rotation of shaft 18 that occurs during dispensing of fluid from prefilled syringe 14.

Specifically, when assembled, sleeve assembly 104 is received within channel 64 of handle body 16 such that shaft 18 passes through both sleeve assembly 104 and handle body 16. Cylindrical sleeve 106 is rigidly coupled to handle body 106 such that shaft 18 is allowed to rotate relative to both sleeve assembly 104 and handle body 16 to advance the head of the plunger of prefilled syringe 14. As shaft 18 is rotated relative to sleeve assembly 104, each pin 110 becomes sequentially aligned with axial groove 112. When one of pins 110 becomes aligned with groove 112, the elasticity of band 108 snaps the aligned pin 110 forward into groove 112 bringing a radially inwardly facing surface of pin 110 into sharp contact with the radially outward facing surface of groove 112. The sharp contact between pin 110 and groove 112 generates noise and/or vibration that is detected by the user. Because pins 110 are evenly spaced around sleeve 106, each time a pin 110 aligns with groove 112 the noise and/or vibration indicates that a predetermined amount of rotation of shaft 18 has been reached. In the embodiment shown, sleeve assembly 104 includes eight evenly space pins resulting in an indication of rotation after every ⅛ revolution (45 degree) of shaft 18.

Referring back to FIG. 2A, driver 12 includes knob 20. Knob 20 is configured to be coupled to upper end 90 of shaft 18 and provides a handle or grip that the user holds while manually rotating shaft 18. Knob 20 includes an outer, circumferential surface 120 that includes knurls that facilitate gripping by the user. The lower surface of knob 20 includes a mounting bore 122 that is sized to receive upper end 90 of shaft 18. In the embodiment shown, knob 20 is rigidly coupled to shaft 18 by a press-fit engagement between shaft 18 and bore 122 such that rotation of knob 20 imparts the same rotation to shaft 18. In other embodiments, other attachment structures or mechanisms may be used. For example, knob 20 may be coupled to shaft 18 via a fastener such as a screw or bolt. As discussed in more detail below, the outer axial or upper end surface 124 may be configured to display or hold label information that may be used to identify the contents of prefilled syringe 14.

Referring to FIG. 3, a detailed view of the attachment between driver 12 and prefilled syringe 14 is shown. Generally, driver 12 engages prefilled syringe 14 in two areas. Shaft 18 engages plunger 34 of prefilled syringe 14 such that rotation of shaft 18 translates into both axial and rotational movement of plunger 34. Handle body 16 rigidly engages outer syringe body 26 such that handle body 16 and outer syringe body 26 are fixed together allowing shaft 18 and plunger 34 to rotate relative to both handle body 16 and outer syringe body 26.

Referring first to the engagement between shaft 18 and plunger 34 shown in FIG. 3, plunger shaft 38 is received within slots 96 and void 102 (both shown in FIG. 2E) such that prongs 98 surround each right angle segment of plunger shaft 38. As noted above, each prong 98 includes an engagement surface, shown as axial facing surface 100, that engages the upper surface or face of plunger head 36. The engagement between surfaces 100 of prongs 98 and plunger head 36 is tight enough that rotation of shaft 18 translates into rotation of plunger head 36 (and plunger shaft 34).

In the embodiment shown, the only contact between shaft 18 and plunger 34 is the contact between surfaces 100 of prongs 98 and the upper surface of plunger head 36. In such an embodiment, the circumferential dimension of each slot 96 between prongs 98 is large enough that there is a gap between each segment of plunger shaft 38 and the circumferential facing surfaces of prongs 98. Thus, the circumferential surfaces of prongs 98 do not contact the outer surface of shaft 38. By providing for only axial contact between shaft 18 and plunger head 36, precise movement of plunger head 36 is provided which in turn provides precise control of the volume of substances dispensed from prefilled syringe 14. Further as shown in FIG. 3, the outer diameter (i.e., the radial dimension) of prongs 98 is less than inner diameter of syringe body 26 such that there is a gap between the outer surface of prongs 98 and the inner surface of syringe body 26. This gap eliminates potential contact that may otherwise impede the transmission of motion from shaft 18 to plunger head 36.

Referring to both FIG. 3 and FIG. 4, the structures providing the rigid engagement between handle body 16 and syringe body 26 are shown. As noted above, handle body 16 includes a first slot 80 and second slot 82 on opposing surfaces of handle body 16 that allow entry of syringe head 46 into attachment structure 76 of handle body 16. As shown best in FIG. 4, attachment structure 76 includes a first engagement surface 130 and a second engagement surface 132 formed within handle body 16. First engagement surface 130 faces upper end 62 of handle body 16. Second engagement surface 132 opposes first engagement surface 130 and faces lower end 60 of handle body 16. As shown, second engagement surface 132 is shown positioned between first engagement surface 130 and upper end 62 such that second engagement surface 132 is above surface 130 in the orientation of FIG. 4.

First engagement surface 130 and second engagement surface 132 define a gap 134 positioned between the two engagement surfaces. The height of gap 134 is such that a friction fit is provided between engagement surfaces 130 and 132 and the upper and lower surfaces of syringe head 46 following coupling of prefilled syringe 14 to driver 12. In one embodiment, surfaces 130 and 132 may be slightly tapered such that the axial dimension of gap 134 decreases toward the center of handle body 16. In this embodiment, the tapered engagement surfaces 130 and 132 provide a friction fit engagement with the surfaces of syringe head 46. The friction fit is rigid such that handle body 16 is fixed relative to syringe body 26 during rotation of shaft 18, and the friction fit is reversible such that when prefilled syringe 14 is empty, driver 12 may be detached (e.g., by application of manual force in a direction opposite from the force used to couple syringe 14 to drive 12) from prefilled syringe 14 allowing driver 12 to be reused attached to a second prefilled syringe 14.

In various embodiments, handle body 16 is configured to provide a twist-lock between prefilled syringe 14 and handle body 16. In the particular embodiment shown, prefilled syringe 14 is attached by inserting the center of syringe body 26 in the gap 136 provided between opposing sections of flared collar 78 in a rotational orientation such that the long axis of syringe head 46 is angled relative to an axis defined by slots 80 and 82 (e.g., a horizontal axis in the orientation of FIG. 4). Prefilled syringe 14 is moved axially (e.g., upward in the orientation of FIG. 4) toward engagement surface 132 such that the axial position of syringe head 46 aligns with the axial position of slots 80 and 82. With syringe head 46 in place adjacent slots 80 and 82, prefilled syringe 14 is rotated clockwise such that one wing of syringe head 46 passes through slot 80 into gap 134 and the other wing of syringe head 46 passes through slot 82 into gap 134. Prefilled syringe 14 is rotated until the long axis of syringe head 46 is substantially parallel to the axis defined by slots 80 and 82 (e.g., a horizontal axis in the orientation of FIG. 4). In this embodiment, engagement surfaces 130 and 132 are shaped such that when the long axis of syringe head 46 is substantially parallel to the axis defined by slots 80 and 82, syringe head 46 is rigidly engaged between engagement surfaces 130 and 132.

Each engagement surface 130 and 132 is divided in to two portions located on opposite sides of central passage 64 of handle body 16. This radial symmetry provides that central passage 64 is aligned with the central bore and the plunger of prefilled syringe 14 following attachment. Axial alignment between central passage 64 and the central bore and the plunger of prefilled syringe 14 allows shaft 18 to properly engage syringe head 36 following coupling of driver 12 to syringe 14.

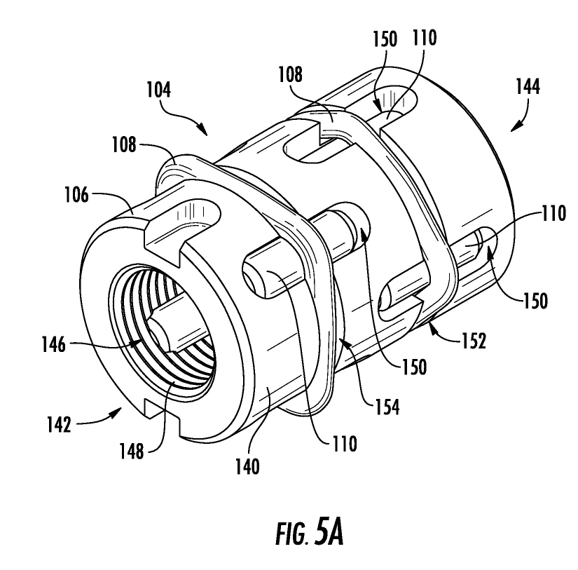

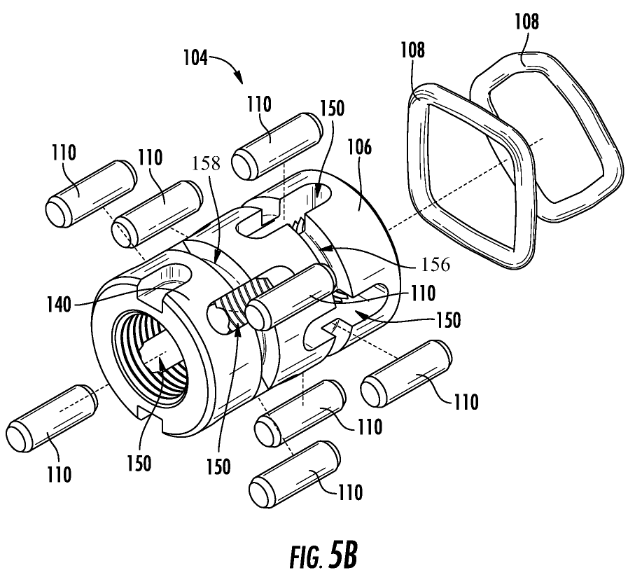

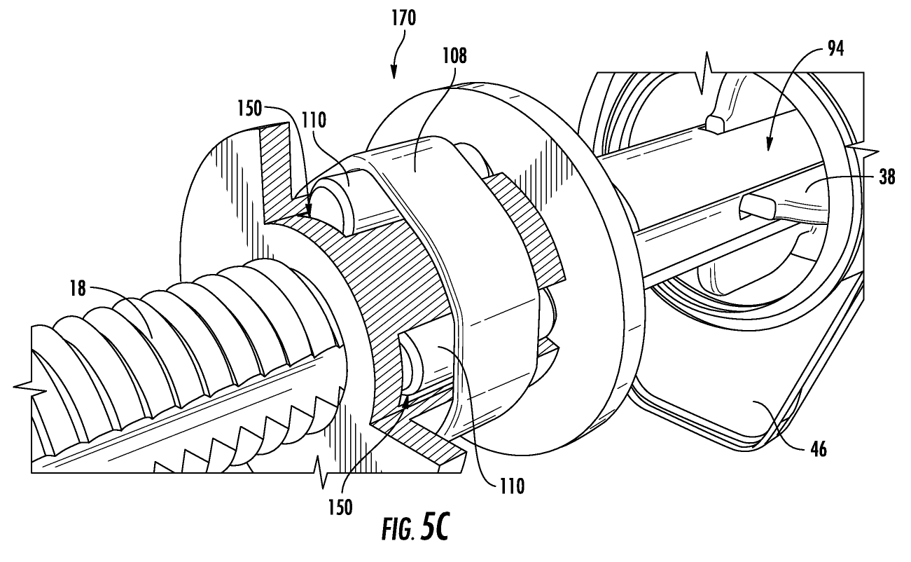

Referring to FIG. 5A and FIG. 5B, assembled and exploded views, respectively, of sleeve assembly 104 is shown according to an exemplary embodiment. Cylindrical sleeve 106 includes a generally cylindrical sidewall 140, an upper end 142, a lower end 144 and a bore 146 extending through sleeve 106 between upper end 142 and lower end 144. The inner surface 148 of sleeve 106 is threaded to mate with the threading of shaft 18. As noted above, shaft 18 is threaded through sleeve 106, and sleeve 106 is rigidly fixed relative to the handle body 16, allowing shaft 18 to be advanced through both sleeve 106 and handle body 16. In other embodiments, sleeve assembly 104 is not a separate component attached shaft 18 but is an indicator integrally formed with the inner structure of handle body 16.

In the embodiment shown in FIG. 5A and FIG. 5B, sleeve assembly 104 is configured to provide an indication of rotation every ⅛ of a rotation. Thus, sleeve assembly 104 includes eight pins 110, and cylindrical sleeve 106 includes eight openings 150 evenly spaced around the circumference of sleeve 106 for receiving pins 110. Openings 150 are radial openings extending through cylindrical sidewall 140. Pins 110 are located within the openings such that the radial inner most surfaces of pins 110 are permitted to contact the outer surface of shaft 18. Elastic bands 108 are in contact with each pin 110 such that when axial groove 112 of shaft 18 is located beneath a given pin 110, the band 108 pushes the pin inward creating an audible and/or tactile indication that shaft 18 has been rotated 45 degrees.

In the embodiment shown, cylindrical sleeve 106 includes a lower row 152 of four openings and an upper row 154 of openings 150. The openings of lower row 152 are rotated 45 degrees relative to the openings of upper row 154, resulting in one opening 150 every 45 degrees around the circumference of sleeve 106. Using two staggered rows of openings allows for the use of larger pins 110 which provides for a louder audible indication of rotation and a stronger tactile indication of rotation than if all eight pins were positioned around sleeve 106 in a single row.

In the embodiment shown with two rows of openings and pins, sleeve assembly 170 includes two biasing elements, shown as two elastic bands 108. In this embodiment, sleeve 106 includes two circumferential channels, shown as lower channel 156 and upper channel 158. Lower channel 156 is positioned to axially bisect the openings of lower row 152 and upper channel 158 is positioned to axially bisect the openings of upper row 154. Lower channel 156 receives one of the elastic bands 108, and upper channel 158 receives the other elastic bands 108. Lower and upper channels 156 and 158 act to retain elastic bands 108 in the proper position in engagement with pins 110.

In the various embodiments, the materials of pins 110 and shaft 18 are selected to provide audible and/or tactile indications of rotation. In one embodiment, both pins 110 and shaft 18 are made from metal to provide an easily detected sound and vibration upon impact of shaft 18 by pin 110. In particular, pins 110 and shaft 18 may be a nonreactive metallic metal such as stainless steel or brass. In other embodiments, other materials may be used as needed for a particular application.

Referring to FIG. 5C, in other embodiments, the indicating element is configured to provide indications of rotation at other amounts of rotation to provide for the appropriate dispensed amount of fluid needed for a desired application. For example in FIG. 5C, a sleeve assembly 170 is shown having four pins 110 and four openings 150 located every ninety degrees around the circumference of the sleeve assembly. In this embodiment, sleeve assembly 170 provides an indication of the amount of rotation every ¼ rotation (i.e., 90 degrees). Sleeve assembly 170 may be used for a driver intended for use with a prefilled syringe 14 that requires twice as much fluid to be delivered per application as compared to sleeve assembly 104. Further, sleeve assembly 170 may be used for a driver in which shaft 18 has a lower thread pitch which requires a greater rotation of shaft 18 to obtain the desired amount of delivered fluid.

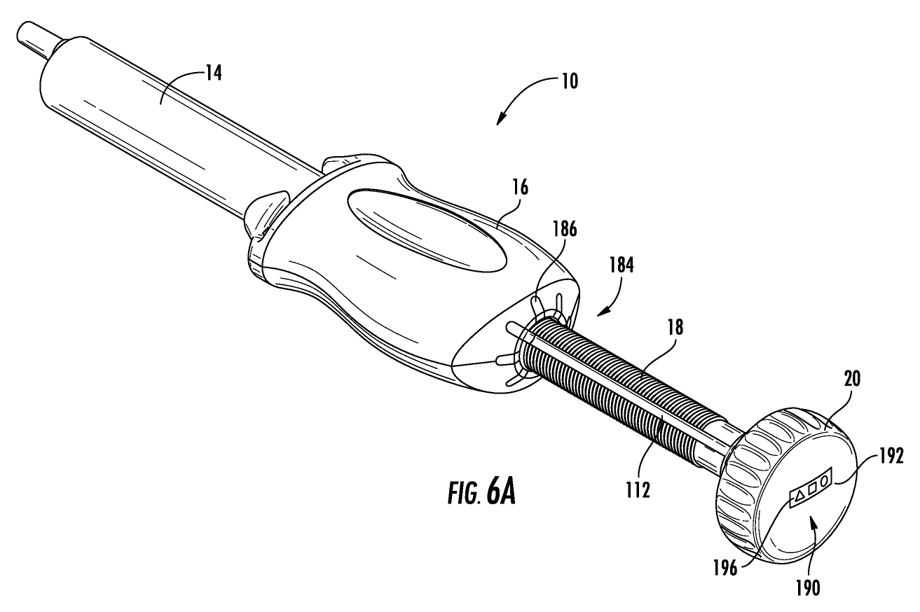

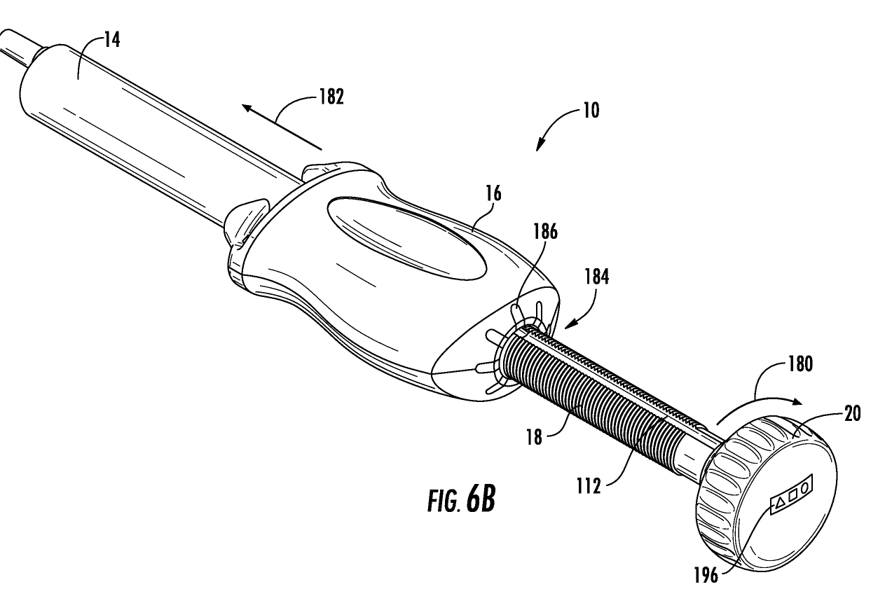

Referring to FIG. 6A and FIG. 6B, operation of dispenser 10 to dispense fluid is shown according to an exemplary embodiment. Both FIG. 6A and FIG. 6B show rear perspective views of driver 12. FIG. 6A shows driver 12 in a first position relative to syringe 14 in which handle body 16 is rigidly fixed to syringe body 26, and shaft 18 is rigidly fixed to syringe plunger 34.

To dispense a predetermined amount of fluid, shaft 18 is rotated clockwise in the direction shown by arrow 180 to a second position as shown in FIG. 6B. As shaft 18 rotates through the threaded channel of sleeve 106, shaft 18 causes both rotational and axial movement of plunger 34 of prefilled syringe 14, shown by arrow 182. Axial movement of plunger 34 decreases the volume of the contents chamber 44 resulting in the dispensing of the predetermined amount of fluid from prefilled syringe 14. Because the volumes being dispensed are small, the rotational movement of plunger 34 allows for more precise dispensing of fluid by overcoming the static friction via rotational movement of plunger 34. Thus, rotation of plunger 34 decreases the likelihood of inadvertent over-dispensing of fluid and abrupt movement of plunger 34 that could otherwise decrease the accuracy of the dispensed volume.

In various embodiments, dispenser 10 is configured to accurately dispense fluid as needed for a particular application. In particular, driver 12 and prefilled syringe 14 are configured such that a predetermined amount of fluid is dispensed from syringe 14 when shaft 18 is rotated a predetermined distance. In various embodiments, the predetermined rotational amount or distance is between 0 degrees and 180 degrees of rotation, and in some such embodiments, the predetermined amount of fluid dispensed during rotation is between 5 microliters and 50 microliters.

In other embodiments, the predetermined rotational amount is greater than or equal to ⅛ of a rotation and is less than or equal to a ½ of a rotation, and in some such embodiments, the predetermined amount of fluid to be dispensed is between 10 microliters and 40 microliters. In other embodiments, the predetermined rotational amount is greater than or equal to an ⅛ of a rotation and is less than or equal to a ¼ of a rotation, and the predetermined amount of fluid to be dispensed is between 10 microliters and 30 microliters. In one specific embodiment, the rotational amount is equal to an ⅛ of a rotation, and the predetermined amount of fluid to be dispensed is about 12 microliters (e.g., 12 microliters plus or minus one microliter). In another specific embodiment, the rotational amount is equal to a ¼ of a rotation, and the predetermined amount of fluid to be dispensed is about 24 microliters (e.g., 12 microliters plus or minus one microliter). In other embodiments, driver 12 is configured to dispense between 0.2 and 0.4 microliters per degree of rotation, specifically to dispense between 0.2 and 0.3 microliters per degree of rotation, and more specifically to dispense between 0.25 and 0.27 microliters per degree of rotation. In one specific embodiment, driver 12 is configured to dispense between 0.26 and 0.27 microliters per degree of rotation. It should be understood that given a particular desired delivery amount, the predetermined amount of rotation needed is a function of the thread pitch of shaft 18 (which correlates to the distance of axial movement of the shaft per degree of rotation) and a function of the area of the surface of the plunger head that is in contact with the fluid in prefilled syringe 14.

Referring further to FIG. 6A and FIG. 6B, in one embodiment, driver 12 includes a second indicating structure, shown as visual indicator 184. Visual indicator 184 includes a series of evenly spaced marks 186 surrounding shaft 18. Marks 186 are positioned on the outer surface of the upper end of handle body 16. Marks 186 may be applied to the outer surface, for example, via paint or applied label. In other embodiments, marks 186 may be permanent marks molded into the material of body 16. The number of marks 186 spaced around shaft 18 is determined by the predetermined rotational amount for a particular driver 18, and in this embodiment, there are eight marks 186 such that the predetermined rotational amount is an ⅛ of rotation. However, visual indicator 184 may provide indication of any of the predetermined rotational amounts discussed herein. In this embodiment, groove 112 of shaft 18 acts as an alignment mark providing a visual indication that shaft 18 has been rotated the predetermined rotational amount when groove 112 aligns with the next clockwise mark 186. Driver 12 may include only visual indicator 184, only an audible/tactile indicator, such as sleeve assembly 104, or both a visual indicator and an audible/tactile indicator.

Referring further to FIG. 6A and FIG. 6B, knob 20 includes a label area 190 located on the axial surface away from shaft 18. Because driver 12 is able to be used with multiple prefilled syringe 14, label area 190 may include a label or indicia 196 that indicates the contents of the prefilled syringe currently attached to driver 12. Knob 20 may include a cover 192 that snap fits onto knob 20 covering label 190 to protect the label information contained in label 190. In this embodiment, the label held by label area 190 is coupled to the upper end of shaft 18 via label area 190 of knob 20 such that the label is substantially perpendicular to the longitudinal axis of the shaft. After attachment to prefilled syringe 14, driver 12 may be stored upright in a rack, and the positioning of label area 190 substantially perpendicular to the longitudinal axis of shaft 18 is such that the label in label area 190 is facing generally upward when the driver is stored upright. This positioning may help ensure that label area 190 is readable when dispenser 10 is in storage.

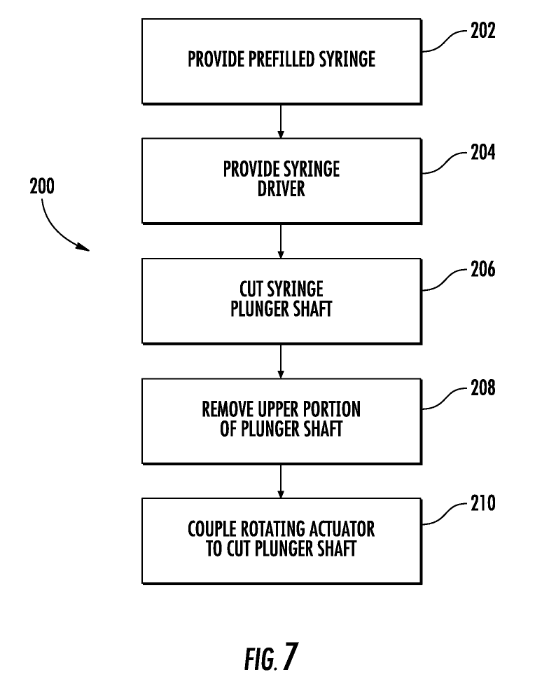

Referring to FIG. 7, a flow-diagram of a method 200 of modifying a prefilled syringe is shown according to an exemplary embodiment. At step 202, a prefilled syringe is provided. In one embodiment, the prefilled syringe provided is prefilled syringe 14 discussed herein. At step 204, a driver is provided that includes a rotating actuator (e.g., shaft 18) that imparts both rotational and axial movement to the syringe plunger to dispense fluid from the prefilled syringe. In one embodiment, the driver is driver 12 discussed herein. At step 206, the plunger shaft of the prefilled syringe is cut at a position between the plunger seal and the plunger top. At step 208, the portion of the shaft of the prefilled syringe above the cut is removed. After this step the cut surface of the plunger shaft is the upper most surface of the plunger shaft. At step 210, the rotating actuator of the driver is coupled to the plunger such that upon operation the rotating actuator imparts both rotational and axial movement to the plunger seal to dispense the predetermined amount of fluid from the prefilled syringe. In one embodiment of the method shown in FIG. 7, step 210 further includes rigidly coupling the driver to the syringe body such that the rotating actuator can rotate the plunger within the syringe body without also rotating the syringe body. In various embodiments, method 200 further includes the steps of assembling the driver by rigidly coupling sleeve assembly 104 to the inner surface of handle body 16 and coupling shaft 18 to the threaded inner surface of sleeve assembly 104.

In various embodiments, a kit for dispensing liquid from a prefilled syringe is provided. In one embodiment, the kit may include one or more driver 12 and one or more prefilled syringe 14. In one such embodiment, the kit includes the same number of drivers 12 as prefilled syringes 14. In such embodiments, each prefilled syringe 14 contains a different substance. In various embodiments, the kit includes a cutting device. In various embodiments, the kit includes instructions setting forth the steps of method 200 discussed below. In one such embodiment, the instructions are coupled to the shaft of the prefilled syringe in a manner that is visible during cutting of the plunger shaft and during attachment of driver 12 to the prefilled syringe. For example, the instructions are attached to the portion of the syringe plunger between the cut and the plunger top.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A driver for engaging a sealed, prefilled syringe, the prefilled syringe having a syringe body, a flange extending from an upper end of the syringe body, and a plunger received within the syringe body, the driver comprising :
a handle body, the handle body comprising:
a first end;
a second end opposite the first end;
a central passage extending through the body from the first end to the second end, and
an attachment structure located at the second end, the attachment structure configured to rigidly attach to the flange of the prefilled syringe such that the syringe body is held in a fixed position relative to the handle body;
a threaded inner surface located within the central passage; and
a shaft configured to extend through the central passage, the shaft comprising:
a first end;
a second end opposite the first end;
an engagement section adjacent the second end configured to engage a portion of the syringe plunger, the engagement section including a first slot formed in the shaft configured to receive a portion of the syringe plunger; and
a threaded outer surface portion configured to engage the threaded inner surface;
wherein rotation of the shaft relative to the handle body is configured to cause advancement of the shaft through the central passage imparting both rotational movement and axial movement to the plunger of the prefilled syringe.

2. The driver of claim 1 further comprising:
an indicator configured to generate an indication when the shaft has been rotated a predetermined rotational amount;
wherein the threaded outer surface portion includes a thread pitch configured such that rotation of the shaft the predetermined rotational amount causes the plunger of the prefilled syringe to move a predetermined axial distance resulting in a predetermined amount of fluid to be dispensed from the prefilled syringe.

3. The driver of claim 2 wherein the predetermined rotational amount is between 0 degrees and 180 degrees and the predetermined amount of fluid to be dispensed is between 5 microliters and 50 microliters.

4. The driver of claim 2 wherein the predetermined rotational amount is greater than or equal an ⅛ of a rotation and is less than or equal to a ½ of a rotation, wherein the predetermined amount of fluid to be dispensed is between 10 microliters and 30 microliters.

5. The driver of claim 2 wherein the indicator is configured to generate an audible indication that the shaft has been rotated the predetermined rotational amount.

6. The driver of claim 5 wherein the indicator comprises:
a surface in contact with the outer surface of the shaft; and
a biasing element biasing the surface into contact with an outer surface of the shaft;
wherein the biasing element is configured to generate movement of the surface when the predetermined rotational amount is reached and the movement of the surface generates the audible indication.

7. The driver of claim 5 further comprising a sleeve located within the central passage of the handle body, the sleeve comprising:
a first end;
a second end opposite the first end;
a generally cylindrical sidewall;
a threaded bore extending through the sleeve from the first end to the second end, wherein the shaft extends through the bore, and wherein the threaded bore is the threaded inner surface located within the central passage of the handle body;
a plurality of openings extending through the sidewall;
a plurality of rigid bodies, each rigid body located within one of the openings and moveable radially within the opening;
a biasing element engaging the rigid bodies, wherein the biasing element is configured to generate a radially inwardly directed force pushing the rigid bodies radially toward the threaded bore;
wherein the shaft includes a depression formed in the threaded outer surface portion of the shaft;
wherein the sleeve is rigidly fixed to the handle body such that the sleeve does not move relative to the handle body as the shaft rotates;
wherein, as the shaft rotates, the depression is sequentially aligned with each rigid body, and when the depression is aligned with one of the rigid bodies, the biasing element pushes the rigid bodies into contact with depression generating the audible indication that the predetermined rotational amount is reached.

8. The driver of claim 2 wherein the indicator comprises a series of evenly spaced marks located on the outer surface of the handle body, wherein the shaft includes an alignment mark, wherein, during rotation of the shaft, indication that the shaft has been rotated a predetermined rotational amount is provided when the alignment mark of the shaft aligns with the next of the evenly spaced marks.

9. The driver of claim 1 wherein the attachment structure of the handle body comprises:
a first engagement surface formed within the handle body, the first engagement surface facing the first end of the handle body;
a second engagement surface formed within the handle body, the second engagement surface facing the first engagement surface and is located between the first engagement surface and the first end of the body;
a gap positioned between the first and second engagement surfaces configured to receive the flange of the prefilled syringe, wherein the first and second engagement surfaces are configured to engage opposite surfaces of the flange providing a friction fit attachment between the handle body and the flange of the prefilled syringe.

10. The driver of claim 9 wherein the attachment structure of the handle body comprises:
a first slot opening formed in a front surface of the handle body and extending laterally along the front surface of the handle body;
a second slot opening formed in a rear surface of the handle body and extending laterally along the rear surface of the handle body, the first and second slot openings providing entry of the flange of the prefilled syringe into the gap;
wherein the attachment structure is configured such that prefilled syringe is rotated relative to the handle body to bring the first and second engagement structures to bring into engagement with the flange of the prefilled syringe.

11. The driver of claim 1 wherein the engagement section of the shaft includes a second slot positioned perpendicular to the first slot configured to receive a cross-shaped portion of the plunger of the prefilled syringe.

12. The driver of claim 1 further comprising:
a sealed, prefilled syringe, wherein the sealed, prefilled syringe contains an allergen test solution; and
a label coupled to the first end of the shaft, the label substantially perpendicular to the longitudinal axis of the shaft and including indicia identifying the allergen test solution.

13. A system for dispensing fluid from a prefilled syringe, the system comprising:
a prefilled syringe comprising:
a syringe body including an upper end, a lower end opposite the upper end, and a central cavity extending from the upper end to the lower end;
a plunger including a plunger head received within the central cavity of the syringe body and a plunger shaft coupled to the plunger head and extending from the plunger head toward the upper end, wherein the plunger head forms a seal within the central cavity defining a contents chamber; and
a fluid located within the contents chamber; and
a driver comprising:
a driver body rigidly attached to the upper end of the prefilled syringe such that the syringe body is held in a fixed position relative to the driver body; and
a rotating actuator engaging the plunger of the prefilled syringe configured to impart both rotational and axial movement to the plunger to dispense the fluid from the prefilled syringe.

14. The system of claim 13 wherein the rotating actuator includes a void and an engagement surface, wherein at least a portion of the plunger shaft is received within the void and the engagement surface engages an upper surface of the plunger head.

15. The system of claim 14 wherein the only contact between the rotating actuator and the plunger is between the engagement surface of the rotating actuator and the upper surface of the plunger head.

16. The system of claim 13 wherein the driver body includes a central channel and the rotating actuator is a threaded shaft extending through the central channel of the driver body, wherein the threaded shaft rotates relative to the driver body and to the syringe body.

17. The system of claim 13 wherein the area and shape of a surface located at the upper end of the plunger shaft is the same as the cross-sectional area and shape of at least a portion of the shaft.

18. The system of claim 13 wherein the fluid is an allergen test solution, the system further comprising a label coupled to the rotating actuator including indicia identifying the allergen test solution.

19. A method of modifying a prefilled syringe comprising:
providing a prefilled syringe comprising:
a syringe body including a first end, a second end opposite the first end, and a central cavity extending from the first end to the second end;
a plunger including a plunger seal, plunger top and a plunger shaft extending between the plunger seal and the plunger top; and
a fluid located within the central cavity;
providing a driver including a rotating actuator, the driver configured to impart both rotational and axial movement to the plunger to dispense the fluid from the prefilled syringe;
cutting the plunger shaft at a position between the plunger seal and the plunger top;
removing the portion of the plunger shaft above the cut including the plunger top;
coupling the rotating actuator of the driver to the plunger.

20. The method of claim 19 further comprising rigidly coupling the driver to the syringe body such that the rotating actuator rotates the plunger within the syringe body.
